(19) United States
(12) Patent Application Publication
(10) Pub. No.: US 2006/0013785 A1
(43) Pub. Date: Jan. 19, 2006

(54) Hairstyling gels with light protective action for skin and hair

(76) Inventors: Dirk Lauscher, Darmstadt (DE); Gabriele Hess, Erzhausen (DE); Susanne Birkel, Darmstadt (DE); Rolf Thiel, Darmstadt (DE); Michael Franzke, Rossdorf (DE)

Correspondence Address: STRIKER, STRIKER & STENBY, 103 EAST NECK ROAD, HUNTINGTON, NY, 11743, US

(21) Appl. No.: 11/180,024

(22) Filed: Jul. 12, 2005

(30) Foreign Application Priority Data
Jul. 15, 2004 (DE) 10 2004 034265.2

(51) Int. Cl. A61K 8/49

(52) U.S. Cl. 424/70.9

(57) ABSTRACT

The hairstyling agent in gel form contains a combination of at least one gel former, more than two percent by weight of at least one UV filter and at least one hair-fixing polymer. The hairstyling gel preferably is in optically clear form and can simultaneously have hair fixing action, light protective action for the skin and light protective action for the hair or dyed hair colors. Preferred UV filters have both a benzimidazole group and a sulfonic acid group.

### BACKGROUND OF THE INVENTION

The subject matter of the present invention is a hairstyling gel with light protective action for skin and hair containing a gel former, more than 2% by weight of a UV filter and at least one hair-fixing polymer.

Hair treatment compositions in the form of gels are used in order to impart hold and to fix human hair, in order to stabilize a hairstyle or hairstyle properties or to improve hair structure. Optically clear or at least nearly optically clear translucent or transparent products are preferred. Products, which have additional uses extending beyond their basic action, e.g. a sunscreen action for scalp skin coming in contact with the product or light protective action for treated hair or a light protective action for dyed hair, are desirable. Hairstyling gels are known, which contain small amounts of light protective substances to protect the product mass. However these amounts are too small in order to be able to provide sufficient UV protection for the skin. These types of hair gels with small amounts of UV filters are described, e.g. in WO 01/13884, U.S. Pat. No. 5,843,415 or EP 867 167. It is not possible to increase the amount of the UV filters in these compositions without more, since it is expected that this can impair the product stability of clear gels, especially in regard to clarity, viscosity properties, consistency, undesired coloration or incompatibilities with other ingredients. Many highly effective UV filters are e.g. hydrophobic in nature. The use of larger amounts of hydrophobic water-insoluble components in the conventional water-based styling gels can impair optical clarity of these products. Hydrophilic UV filters can be ionic or nonionic. Ionic components usually have a negative viscosity-reducing influence on the viscosity of the gel because of their electrolytic character, when they are used in high concentrations, since the gel formers usually used in the styling gels are frequently sensitive to electrolytes. Moreover difficulties are expected, when hydrophobic substances, such as perfume oils in optically clear form, should be emulsified in a gel that is highly loaded with electrolytes. Hydrophobic perfume oils can be worked into conventional aqueous gels by means of suitable emulsifiers in optically clear form. However this is not possible without more when the gels are highly loaded with electrolytes because turbidity, phase separation and/or non-uniformities can occur. Nonionic hydrophilic UV filters, e.g. those with polyethylene glycol chains, can lead to undesirable gel-forming discoloration during storage, when they are used in larger amounts in clear gels.

### SUMMARY OF THE INVENTION

It is an object of the present invention to provide hairstyling gels with three-fold action, namely hair-fixing action, light protective action for hair and light protective action for skin.

It is also an object of the present invention to provide optically clear or at least nearly clear or translucent or transparent gels, which have sufficient stability regarding consistency and optical impression during longer storage times.

According to the invention these hairstyling gels contain a combination of
(A) at least one gel former;
(B) more than two percent by weight of at least one UV filter and
(C) at least one hair-fixing polymer.

The gel former (A) is preferably contained in the gel according to the invention preferably in an amount of 0.1 to 20 percent by weight or from 0.5 to 10 percent by weight. The UV filter or filters (B) are preferably contained in an amount of up to 15 percent by weight, especially of 3 to 9 percent by weight. The hair-fixing polymer (C) is preferably contained in an amount of from 0.1 to 15 percent by weight, 0.2 to 10 percent by weight or 0.5 to 5 percent by weight.

Optically clear or nearly clear, slightly opaque products, especially gels, in which turbidity is not detectable with the naked eye, are preferred. The light or sun protective factor, SPF, of the gel is preferably at least 4, especially preferably greater than or equal to 6, greater than or equal to 10 or greater than or equal to 15. The SPF can be determined, e.g. according to the standardized methods described in the COLIPA publication, “International Sun Protective Factor Test Method” (2003).

Gel Former

The gel former (A) is preferably a gel-forming, cross-linked polymer, e.g.
a copolymer of at least one first monomer and at least one second monomer, wherein the at least one first monomer is acrylic acid and/or methacrylic acid and the at least one second monomer is selected from the group consisting of esters of acrylic acid and ethoxylated fatty alcohols, e.g. acrylates/palmeth-25 acrylate copolymers, SYNTHALEN® 2000W;
a cross-linked polyacrylic acid, e.g. Carbomer, CARBOPOL® 980, 981, 2001 ETD, 2020 ETD, Carbomer K;
a cross-linked copolymer of at least one first monomer and at least one second monomer, wherein the at least one first monomer is acrylic acid and/or methacrylic acid, and the at least one second monomer is selected from the group consisting of esters of acrylic acid with C₁₀- to C₃₀-alcohols; e.g. acrylates/C10-30 alkylacrylate crosspolymer, PEMULEN® TR1, CARBOPOL® UITREZ® 21;
a copolymer of at least one first monomer and at least one second monomer, wherein the at least one first monomer is acrylic acid and/or methacrylic acid, and the at least one second monomer is selected from the group consisting of esters of itaconic acid and ethoxylated fatty alcohols, e.g. acrylates/steareth-20 itaconate copolymer, acrylates/ceteth-20 itaconate copolymer, STRUCTURE® 2001, STRUCTURE® 3001;
a copolymer of at least one first monomer, at least one second monomer and a third monomer, wherein the at least one first monomer is acrylic acid and/or methacrylic acid, the at least one second monomer is selected from the group consisting of itaconic acid and ethoxylated C₁₀- to C₃₀-alcohol and the third monomer is a C₁- to C₄-aminoalkyl acrylate, for example an acrylates/aminoacrylates/C₁₀- to C₃₀-alkyl PEG-20 Itaconate copolymer, STRUCTURE® Plus;
a copolymer of two or more monomers selected from the group consisting of acrylic acid, methacrylic acid, acrylic acid esters and methacrylic acid esters, e.g. acrylates copolymer, CARBOPOL® Aqua SF-1;
a copolymer of vinyl pyrrolidone and ammonium acryloyldimethyltaurate; e.g. ARISTOFLEX® AVC;
a copolymer of ammonium acryloyldimethyltaurate and at least one monomer selected from the group consisting of methacrylic acid and ethoxylated fatty alcohols, e.g. ammonium acryloyldimethyltaurate/beheneth-25 methacrylate copolymer, ARISTOFLEX® HMB;
a hydroxyalkyl cellulose, e.g. hydroxyethyl cellulose, hydroxypropyl cellulose, NATROSOL® 250 HHR;
hydroxypropyl guar;
glycerylpolyacrylate and glycerylpolymethacrylate, e.g. HISPAGEL® 100, HISPAGEL® 200, LUBRAJEL®;
copolymers of styrene and at least one C₂-, C₃- or C₄-alkylene;
a thickened polyurethane;
a hydroxypropyl starch phosphate, e.g. STRUCTURE® XL;
a polyacrylamide, e.g. SEPIGEL® 305;
a copolymers of maleic acid anhydride and methyl vinyl ether cross-linked with decadiene, e.g. PVM/MA decadiene crosspolymer, STABILEZE® 06;
a copolymer of polyethylene oxide, fatty alcohols and saturated methylenediphenyl diisocyanate (e.g. PEG-150/stearyl alcohol/SMDI copolymer);
a thickener on a natural basis, such as carob or locust bean seed meal; guar gum; xanthen, dehydroxyxanthen; carrageenan, karaya gum; hydrolyzed corn starch.

Preferred gel formers include acrylates/C10-30 alkylacrylate crosspolymer, carbomers, xanthan gum and acrylates/Ceteth-20 itaconate copolymer.

UV Filter

The UV filters (B) are preferably organic compounds, which absorb radiation in the UV-A spectral range (315 to 400 nm) and/or in the UV-B spectral range (280 to 315 nm). Preferred UV filters have at least one acid group, especially at least one —SO₃H group and can be present in the end product in partially or completely neutralized form, i.e. as salts. The salts are e.g. ammonium, alkali or alkaline earth salts, especially sodium salts. UV filters, which have at least one benzimidazole or benzoxazole unit in the molecule, are preferred.

Sulfonic acid substituted benzimidazole and benzoxazole have, e.g. the general formula I: 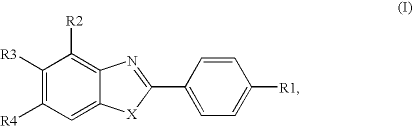
wherein R1 represents a hydrogen, a C₁- to C₁₀-alkoxy group or the following group of formula Ia: 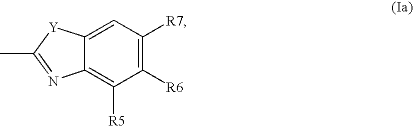
wherein R2 to R7 denote, independently of each other, hydrogen or —SO₃H, wherein at least one of the R2 to R7 groups represents —SO₃H, X and Y each represent either O or NH.

Compounds are especially preferred, in which X and Y (in so far as they are present) equal NH and wherein R2 and R4 are the same and R5 and R7 are the same and different from R3 and R6. Preferred benzimidazoles are, for example, 2-phenyl-1H-benzimidazol-5-sulfonic acid (e.g. Neo HELIOPAN® Hydro), 2,2′-(1,4-phenylen)-bis-(1H-benzimidazol-4,6-disulfonic acid) (e.g. Neo HELIOPAN® AP) and 2,2′-(1,4-phenylen)bis-(1H-benzimidazol-5-sulfonic acid) and its respective salts. A preferred benzoxazole is e.g. 2,2′-(1,4-phenylen)-bis-(oxazol-5-sulfonic acid) and its salts.

Preferably the gel according to the invention contains a combination of at least one UV-A filter with at least one UV-B filter, especially a combination of phenyhlbenzimidazole sulfonic acid or its salts with phenyldibenzimidazole tetrasulfonic acid or its salts.

Hair-Fixing Polymers

Hair-fixing polymers are e.g. those polymers, which are described in the International Cosmetic Ingredient Dictionary and Handbook, 10^{th} Edition, 2004, Vol. 3 under the heading “Hair Fixatives”. Hair-fixing polymers are, especially, those, which are in a position to cause a hair-fixing action, as established e.g. by the usual curl retention measurement, when used in a 0.01 to 5% preparation in a suitable aqueous, alcoholic or aqueous-alcoholic solvent.

Hair-fixing polymers can be non-ionic, anionic, cationic, zwitterionic or amphoteric. When one of the polymeric gel formers or one of the hair-fixing polymers is anionic, preferably the composition does not contain a cationic polymer or only contains it in very small amounts (e.g. less than 1 or less than 0.1 percent by weight), since otherwise undesirable interactions can occur.

Anionic polymers are polymers with anionic or anionizable groups. The term “anionizable group” means an acidic group, e.g. carboxylic acid, sulfonic acid or phosphoric acid groups, which can be deprotonated by means of conventional bases, such as organic amines or alkali or alkaline earth hydroxides.

Polymers with acid groups can be partially or completely neutralized with a basic neutralization agent. Those agents are preferred, in which the acid groups in the polymer are neutralized to 50 to 100%, especially preferably to 70 to 100%. Organic or inorganic bases can be used as the neutralization agent. For example, especially aminoalkanols, such as aminomethylpropanol (AMP), triethanolamine or monoethanolamine are suitable for the bases, but also ammonia, NaOH, KOH, among others, are suitable.

The anionic polymers can be homopolymers or copolymers with monomer units containing acid groups on a synthetic or natural basis, which are copolymerized as needed with comonomers, which contain no acid groups. A sulfuric acid group, a phosphoric acid group and a carboxylic acid group can be used as the acid group. The carboxylic acid group is particularly preferred. Suitable monomers containing acid groups include e.g. acrylic acid, methacrylic acid, crotonic acid, maleic acid, maleic acid anhydride, maleic acid monoesters, especially the mono-C₁ to C₇-alkyl esters of maleic acid, aldehydocarboxylic acids or ketocarboxylic acids. The comonomers not substituted with acid groups include, e.g., acryl amide, methacrylamide, alkyl- and dialkylacrylamides, alkyl- and dialkylmethacrylamides, alkylacrylates, alkylmethacrylates, vinyl capro-actone, vinyl pyrrolidone, vinyl ester, vinyl alcohol, propylene glycol or ethylene glycol, amine-substituted vinyl monomers, such as dialkylamino-alkylacrylates, dialkylaminoalkyl-methacrylates, monoalkylaminoalkylacrylates and monoalkylaminoalkylmethacrylates, in which the alkyl groups of these monomers are preferably C₁- to C₇-alkyl groups, especially preferably C₁- to C₃-alkyl groups.

Suitable polymers with acid groups are especially homopolymers of acrylic acid or methacrylic acid, which are not cross-linked or cross-linked with polyfunctional agents, copolymers of acrylic acid or methacrylic acid with monomers selected from the group consisting of acrylic acid or methacrylic acid esters, acrylamide, methacrylamide and vinyl pyrrolidones, homopolymers of crotonic acid and copolymers of crotonic acid with monomers selected from the group consisting of vinyl esters, acrylic acid esters, methacrylic acid esters, acrylamides and methacrylamides. A suitable natural polymer is, for example, shellac.

Preferred polymers with acid groups are: terpolymers of acrylic acid, alkyl acrylate and N-alkylacrylamide (INCI-name: acrylates/acrylamide copolymer), especially terpolymers of acrylic acid, ethyl acrylate and N-t-butyl-acrylamide; cross-linked or uncrosslinked vinyl acetate/crotonic acid copolymer (INCI name: VA/crotonates copolymer); copolymers of one or more C₁- to C₅-alkylacrylates, especially C₂- to C₄-alkylacrylates and at least one monomer selected from acrylic acid or methacrylic acid (INCI name: acrylates copolymer), e.g. terpolymers from tert.-butylacrylate, ethyl acrylate and methacrylic acid; sodium polystyrene sulfonate; vinyl acetate/crotonic acid/vinyl alkanoate copolymers, e.g. copolymers of vinyl acetate, crotonic acid and vinyl propionate; copolymers of vinyl acetate, crotonic acid and vinyl neodecanoate (INCI names: VA/crotonates/vinyl propionate copolymer, VA/crotonates/vinyl neodecanoate copolymer); aminomethyl propanol-acrylate copolymers; copolymers of vinyl pyrrolidone and at least one other monomer selected from the group consisting of acrylic acid, methacrylic acid, and, as needed, acrylic acid esters and methacrylic acid esters; copolymers of methyl vinyl ether and maleic acid monoalkyl esters (INCI name: ethyl ester of PVM/MA copolymer, butyl ester of PVM/MA copolymer); aminomethylpropanol salts of copolymers of allyl methacrylate and at least one other monomer selected from the group consisting of acrylic acid, methacrylic acid, and, as needed, acrylic acid esters and methacrylic acid esters; cross-linked copolymers of ethyl acrylate and methacrylic acid; copolymers of vinyl acetate, mono-n-butylmaleate and isobornyl acrylate; copolymers of two or more monomers selected from the group consisting of acrylic acid, methacrylic acid, and, as needed, acrylic acid esters and methacrylic acid esters; copolymers of octyl acrylamide and at least one monomer selected from the group consisting of acrylic acid, methacrylic acid, and, as needed, acrylic acid esters and methacrylic acid esters; polyesters of diglycol, cyclohexanedimethanol, isophthalic acid and sulfoisophthalic acid, in which the alkyl groups of the above-named polymers preferably contain 1, 2, 3 or 4 carbon atoms.

Zwitterionic polymers have at least one anionic charge and at least one cationic charge at the same time. Amphoteric polymers have at least one acid group (e.g. carboxylic acid or sulfuric acid group) and at least one base group (e.g. amine group). The acid groups can be deprotonated by a common base, e.g. an organic amine or an alkali metal or alkaline earth metal hydroxide.

Preferred zwitterionic or amphoteric polymers are:

Copolymers formed from alkylacrylamides, alkylaminoalkylmethacrylate and two or more monomers selected from acrylic acid, methacrylic acid, and, as needed, acrylic acid esters and methacrylic acid esters, especially copolymers of octylacryl amide, acrylic acid, butylaminoethylmethacrylate, methylmethacrylate and hydroxypropylmethacrylate (INCI name: octylacrylamide/acrylates/butyl-aminoethyl methacrylate copolymer); copolymers, which are formed from at least one first type of monomer, which has quaternary amino groups and at least one second type of monomer, which has acid groups; copolymers of fatty alcohol acrylates, alkylaminoxide methacrylates and at least one monomer selected from acrylic acid, methacrylic acid, and, as needed, acrylic acid esters and methacrylic acid esters, especially copolymers of lauryl acrylate, stearyl acrylate, ethyl-amine oxide methacrylate and at least one monomer selected from acrylic acid, methacrylic acid and esters thereof as needed; copolymers of methacryloyl-ethylbetaine and at least one monomer selected from methacrylic acid and methacrylic acid esters; copolymers of acrylic acid, methacrylate and methacrylamido-propyltrimethylammonium chloride (INCI: polyquaternium-47); copolymers made from acrylamidopropyltrimethylammonium chloride and acrylates or copolymers made from acrylamide, acrylamidopropyltrimethyl ammonium chloride, 2-amidopropyl-acrylamide sulfonate and dimethylaminopropylamine (INCI: Polyquaternium-43); and oligomers or polymers made from quaternary crotonic betaines or their esters.

In other embodiments of the invention the composition can further comprising a cationic polymer, i.e. a polymer with cationic or cationizable groups, especially primary, secondary or tertiary or quaternary amine groups. The cationic charge density amounts to preferably 1 to 7 meq/g. Suitable cationic polymers preferably contain quaternary amine groups. The cationic polymers can be homopolymers or copolymers, which contain quaternary nitrogen groups in the polymer chain or preferably as substituents on one or more monomers. The monomers containing ammonium groups can be copolymerized with non-cationic monomers. Suitable cationic monomers are unsaturated, radical polymerizable compounds, which contain at least one cationic group, especially ammonium-substituted vinyl monomers include, e.g., trialkylmethacryloxyalkyl ammonium, trialkylacryloxyalkyl ammonium, dialkyldiallyl ammonium and quaternary vinyl ammonium monomers with cyclic, cationic nitrogen containing groups, such as pyridinium, imidazolium or quaternary pyrrolidone, e.g. alkylvinylimidazolium, alkylvinylpyridinium, or alkylvinylpyrrolidone salts. The alkyl groups are preferably lower alkyl groups, especially C₁- to C₇-alkyl groups, preferably C₁- to C₃-alkyl groups.

The monomers containing ammonium groups can be copolymerized with non-cationic monomers. Suitable comonomers are for example acryl amide, methacryl amide, alkyl- and dialkylacrylamide, alkyl- and dialkylmethacryl amide, alkyl acrylate, alkylmethacrylate, vinyl caprolactone, vinyl caprolactam, vinyl pyrrolidone, vinyl ester, e.g. vinyl acetate, vinyl alcohol, propylene glycol or ethylene glycol, wherein the alkyl groups of these monomers preferably have one to seven carbon atoms, especially one to three carbon atoms.

Suitable polymers with quaternary amine groups are, for example, polymers described in the CTFA Cosmetic Ingredient Dictionary under the name “polyquaternium”, such as methylvinylimidazolium chloride/vinyl pyrrolidone copolymer (Polyquaternium-16) or quaternized vinyl pyrrolidone/dimethylamino-ethylmthacrylate copolymer (Polyquaternium-11) and quaternary silicone polymers and/or oligomers, such as silicone polymers with quaternary terminal groups (Quaternium-80).

Preferred cationic polymers on a synthetic basis include: poly(dimethyl-diallylammonium chloride); copolymers of acrylamide and dimethyldiallyl ammonium chloride; quaternary ammonium polymers, formed by reaction of diethyl sulfate and a copolymer of vinyl pyrrolidone and dimethylamino-ethylmethacrylate, especially vinyl pyrrolidone/dimethylaminoethylmethacrylate methosulfate copolymer, (e.g. GAFQUAT® 755 N and GAFQUAT® 734); quaternary ammonium polymers of polyvinyl pyrrolidone and imidazolimine-methochloride (e.g. LUVIQUAT® HM 550); Polyquaternium-35; Polyquaternium-57; polymer from trimethyl ammonium ethylmethacrylate chloride; the terpolymer of dimethyldiallylammonium chloride, sodium acrylate and acrylamide (MERQUAT® Plus 3300); terpolymer of vinyl pyrrolidone, dimethylaminopropylmethacrylamide and methacryloylaminopropyllauryldimethyl ammonium chloride; terpolymer of vinyl pyrrolidone, dimethylaminoethyl-methacrylate and vinyl caprolactam (e.g. GAFFIX® VC 713); vinyl pyrrolidone/methacrylamidopropyltrimethyl ammonium chloride copolymer (GAFQUAT® HS 100); copolymers of vinyl pyrrolidone and dimethylaminoethylmethacrylate; copolymers of vinyl pyrrolidone, vinyl caprolactam and dimethylaminopropyl acrylamide; polyesters or oligoesters built up from at least one first monomer type, which is a hydroxy acid substituted with at least one quaternary ammonium group; substituted dimethylpolysiloxanes with quaternary ammonium end groups.

Suitable cationic polymers, which are derived from natural polymers, are especially cationic derivatives of polysaccharides, e.g. cellulose, starch or guar. Chitosan and chitosan derivative compounds are suitable. Cationic poly-saccharides have the formula II:
G-O—B—N⁺R^{a}R_{b}R_{c}X⁽⁻⁾ (II),
wherein G is an anhydroglucose residue, for example starch or cellulose anhydroglucose;
B is a divalent connecting group, for example, an alkylene, an oxyalkylene, a polyoxyalkylene or hydroxyalkylene;
R^{a}, R^{b} and R^{c} are each, independently of each other, alkyl, aryl, alkylaryl, arylalkyl, alkoxyalkyl or alkoxyaryl with up to 18 carbon atoms respectively, wherein the total number of carbon atoms in R^{a}, R^{b} and R^{c} is at most 20; X is a common counter anion, for example a halogen, acetate, phosphate, nirate or alkyl sulfate, preferably chloride. Preferred cationic celluloses are those marketed under the INCI name, polyquaternium-10 and polyquaternium-24. A suitable cationic guar derivative is e.g. the derivative with the INCI name, guar hydroxypropyltrimonium chloride.

Chitosan, chitosan salts and chitosan derivative compounds are especially preferred as cation-active materials. The chitosan used in the composition of the invention is partially or completely deacetylated. The molecular weights of chitosan can vary over a wide range, for example from 20,000 to 5,000,000 g/mol. A low molecular weight chitosan is, for example, considered to be a chitosan with a molecular weight of from 30,000 to 70,000 g/mol. The molecular weight of the chitosan is preferably above 100,000 g/mol, especially preferably form 200,000 to 700,000 g/mol. The deacetylation degree amounts to from 10 to 99%, especially preferably form 60 to 99%. A preferred chitosan salt is chitosonium pyrrolidone carboxylate, which for example is marketed under the trade name KYTAMER® PC of Amerchol, USA. The chitosan obtained has a molecular weight of about 200,000 to 300,000 g/mol and is deacetylated up to 70 to 85%. Quaternary, alkylated or hydroxyalkylated derivative chitosan compounds, for example, the hydroxyethyl chitosan, hydroxypropyl chitosan or hydroxybutyl chitosan, are suitable in the compositions according to the invention. The chitosans or chitosan derivative compounds should be present in neutralized or partially neutralized form when used in the compositions of the invention. The neutralization degree for the chitosan or the chitosan derivative compounds is preferably at least 50%, especially preferably between 70 and 100%, relative to the number of free base groups. In principle, all cosmetically compatible inorganic or organic acids may be used as neutralization agent, for example formic acid, tartaric acid, malic acid, lactic acid, citric acid, pyrrolidone carboxylic acid, hydrochloric acid, among other.

Preferred cationic polymers on a natural basis include: cationic cellulose derivatives comprising hydroxyethyl cellulose and diallyldimethyl ammonium chloride; cationic cellulose derivates comprising hydroxyethyl cellulose and with trimethyl ammonium substituted epoxide; chitosan and its salts; hydroxyalkyl chitosans and their salts; alkylhydroxyalkyl chitosans and their salts; and N-hydroxyalkyl chitosan alkyl ether.

Nonionic hair-fixing polymer can be of synthetic or natural origin. The term “natural polymers” includes chemically modified polymers of natural origin.

Suitable synthetic nonionic polymers include homopolymers or copolymers, which are built up from at least one of the following monomers: vinyl pyrrolidone, vinyl caprolactam, vinyl esters, such as vinyl acetate, vinyl alcohol, acrylamide, methacrylamide, alkyl- and dialkylacrylamide, alkyl- and dialkylmethacrylamide, alkylacrylate, alkylmethacrylate, propylene glycol or ethylene glycol, wherein the alkyl groups in these monomers preferably have from one to seven carbon atoms, especially preferably from one to three carbon atoms. For example, homopolymers of vinyl caprolactam, of vinyl pyrrolidone or of N-vinylformamide, are especially suitable. Additional suitable synthetic film-forming nonionic hair-fixing polymers are, e.g., copolymerizates of vinyl pyrrolidone and vinyl acetate, terpolymers of vinyl pyrrolidone, vinyl acetate and vinyl propionate, polyacrylamide, polyvinyl alcohols, and polyethylene glycol/polypropylene glycol copolymers. Suitable natural film-forming polymers include e.g. cellulose derivatives, e.g. hydroxyalkyl cellulose.

Preferred nonionic polymers include: polyvinyl pyrrolidone, polyvinyl caprolactam, vinyl pyrrolidone/vinyl acetate copolymers, polyvinyl alcohol, isobutylene/ethylmaleimide/hydroxyethyl maleimide copolymer; copolymers of vinyl pyrrolidone, vinyl acetate and vinyl propionate.

In various embodiments the composition according to the invention contains at least one silicone compound in an amount of preferably from 0.01 to 15 percent by weight, especially preferably from 0.1 to 5 percent by weight, preferably in a clear, e.g., emulsified form as a microemulsion. The silicone compounds include volatile and non-volatile silicone compound and silicones which are soluble and insoluble in the composition. One embodiment of the hair-styling gel according to the invention contains a high molecular weight silicone with a viscosity of 1,000 to 2,000,000, preferably 10,000 to 1,800,000 or 100,000 to 1,500,000, cSt at 25° C. The silicone compounds comprise polyalkylsiloxanes and polyarylsiloxanes, especially with methyl-, ethyl-, propyl-, phenyl-, methylphenyl- and phenylmethyl groups. Polydimethylsiloxanes, polydiethylsiloxanes, polymethylphenylsiloxanes are preferred. Also luster-imparting arylated silicones with an index of refraction of at least 1.46, or at least 1.52, are preferred. The silicone compound comprise, especially substances with the INCI names cyclomethicone, dimethicone, dimethiconol, dimethicone copolyol, phenyl trimethicone, amodimethicone, trimethylsilylamodimethicone, stearyl siloxysilicates, polymethylsilsesquioxanes and dimethicone crosspolymer. Also silicone resin and silicone elastomers, which are highly cross-linked siloxanes, are suitable.

Preferred silicones include: cyclic dimethylsiloxanes, linear polydimethyl-siloxanes, block polymers of polydimethylsiloxane and polyethylene oxides and/or polypropylene oxides, polydimethyl siloxanes with polyethylene oxide or polypropylene oxide terminal or side chain groups, polydimethyl siloxanes with terminal hydroxy groups, phenyl-substituted polydimethyl-siloxanes, silicone emulsions, silicone elastomers, silicone waxes, silicone gums and amino-substituted silicones.

In one embodiment the hairstyling gels contain from 0.01 to 20, preferably from 0.05 to 10, especially preferably from 0.1 to 5, percent by weight of at least one hair-conditioning additive. The at least one hair-conditioning additive is selected from the group consisting of betaine, panthenol, panthenyl ethyl ether, sorbitol, protein hydrolyzate and plant extracts.

In one embodiment the composition according to the invention contains from 0.01 to 20, especially preferably from 0.05 to 10, most preferably from 0.1 to 5, percent by weight of at least one surfactant. The surfactant can be nonionic, anionic, cationic or zwitterionic and especially can act as an emulsifier or solvating agent for hydrophobic additive ingredients, such as perfume oils. Preferred gels contain at least one perfume oil in an optically clear emulsified form.

Non-ionic surfactants include, e.g.:
ethoxylated fatty alcohols, ethoxylated fatty acids, ethoxylated fatty acid glycerides or alkylphenols, especially addition products of 2 to 30 mol ethylene oxide and/or 1 to 5 mol propylene oxide to C₈- to C₂₂-fatty alcohols, to C₁₂- to C₂₂-fatty acids or to alkylphenols with 8 to 15 carbon atoms in their alkyl groups;
C₁₂- to C₂₂-fatty acid monoesters and diesters of addition products of 1 to 30 mol ethylene oxide with glycerol;
addition products of 5 to 60 mol of ethylene oxide with castor oil or hardened (hydrogenated) castor oil; and
fatty acid sugar esters, especially esters of saccharoses and one or two fatty acids with 8 to 22 carbon atoms; e.g. compounds with the INCI names: sugar cocoate, sucrose dilaurate, sucrose distearate, sucrose laurate, sucrose myristate, sucrose oleate, sucrose palmitate, sucrose ricinoleate, sucrose stearate;
esters of sorbitan and one, two or three fatty acids with 8 to 22 carbon atoms and with an ethoxylation degree of 4 to 20;
polyglyceryl fatty acid esters, especially of one, two or several fatty acids with 8 to 22 carbon atoms and polyglycerol with preferably 2 to 20 glyceryl units; and
alkyl glucosides, alkyl oligoglucosides and alkylpolyglucosides with C₈- to C₂₂-alkyl groups, e.g. decyl glucosides or lauryl glucoside.

Anionic surfactants include e.g. salts and esters of carboxylic acids, alkyl ether sulfates and alkyl sulfates, fatty alcohol ether sulfates, sulfonic acids and their salts (e.g. sulfosuccinate or fatty acid isethionate), phosphoric acid esters and their salts, acylamino acids and their salts. A complete description of these anionic surfactants can be found in the publication “FIEDLER—Dictionary of Auxiliary Ingredients”, Volume 1, fifth Edition (2002), pp. 97 to 102, which is hereby incorporated herein by reference. Preferred anionic surfactants are the mono-, di- and/or triesters of phosphoric acid with addition products of 2 to 30 mol ethylene oxide to C₈- to C₂₂-fatty alcohols.

Amphoteric surfactants are, e.g., derivatives of aliphatic quaternary ammonium-, phosphonium- and sulfonium compounds of the following formula III:
R²—Y⁽⁺⁾(R¹)ₓ—CH₂—R³-Z⁽⁻⁾ (III),
wherein R¹ is a straight chain or branched chain alkyl, alkenyl- or hydroxyalkyl group with 8 to 18 carbon atoms and 0 to about 10 ethylene oxide units and 0 to 1 glyceryl unit; Y is a N-, P- or S-containing group; R² is an alkyl- or monohydroxy alkyl group with 1 to 3 carbon atoms; x=1 in the case that Y is a sulfur atom and x=2 when Y is a nitrogen or phosphorus atom; R³ is an alkylene or hydroxyalkylene group with 1 to 4 carbon atoms and Z⁽⁻⁾ represents a carboxylate, sulfate, phophonate or phosphate group.

Other amphoteric surfactants, like betaine are also suitable. For example betaines include C₈- to C₁₈-alkylbetaines, such as cocodimethylcarboxymethyl betaine, lauryldimethylcarboxymethylbetaine, lauryldimethylalphacarboxy-ethylbetaine, cetyldimethylcarboxymethylbetaine, oleyldimethylgammacarboxy-propylbetaine and lauryl-bis-(2-hydroxypropyl)-alphacarboxyethylbetaine; C₈- to C₁₈-sulfobetaines, such as cocodimethylsulfopropylbetaine, stearyl-dimethylsulfopropylbetaine, lauryldimethylsulfoethylbetaine, lauryl-bis-(2-hydroxy-ethyl)sulfopropylbetaine; the carboxyl derivatives of imidazoles; C₈- to C₁₈-alkyl-dimethylammonium acetates, C₈- to C₁₈-alkylalkyldimethylcarbonylmethyl-ammonium salts and C₈- to C₁₈-alkyl fatty acid alkylamidobetaines, e.g. coconut fatty acid amidopropylbetaine and N-coconut fatty acid amidoethyl-N-[2-(carboxymethoxy)-ethyl]-glycerol (CFTA: cocoamphocarboxyglycinate).

Cationic surfactants contain amino groups or quaternized hydrophilic ammonium groups, which carry a positive charge and are represented by the general formula (IV):
N⁺R¹R²R³R⁴X⁽⁻⁾ (IV),
wherein each of R¹ to R⁴, independently of each other, denote an aliphatic group, an aromatic group, an alkoxy group, a polyoxyalkylene group, an alkylamido group, a hydroxyalkyl group, an aryl group or an alkaryl group, each with from 1 to 22 carbon atoms, wherein each of the R¹ to R⁴ groups has at least eight carbon atoms. X⁽⁻⁾ is a cosmetically compatible anion. The anion can be a halogen, acetate, phosphate, nitrate or alkylsulfate anion, but is preferably chloride. The aliphatic groups can also contain cross-linkages or other groups, for example amino groups, besides the carbon atoms and the hydrogen atoms.

For example, suitable cationic surfactants include the chlorides or bromides of alkyldimethylbenzylammonium salts, alkyltrimethylammonium salts, e.g. cetyltrimethylammonium chloride or bromide, tetradecyltrimethylammonium chloride or bromide, alkyldimethylhydroxyethylammonium chloride or bromide, dialkyldimethylammonium chloride or bromide, alkylpyridinium salts, especially lauryl or cetyl pyridinium chloride, alkylamidoethyltrimethylammonium ether sulfate and cationic compounds, such as amine oxides, especially alkylmethylamine oxides or alkylaminoethyldimethylamine oxides. The following are particularly preferred: C₈- to C₂₂-alkyldimethylbenzylammonium compounds, C₈- to C₁₈-alkyltrimethylammonium compounds, especially cetyltrimethylammonium chloride, C₈- to C₁₈-alkyldimethylhydroxyethylammonium compounds, di-(C₈- to C₂₂-alkyl)-dimethylammonium compounds, C₈-C₂-alkyl pyridinium salts, C₈- to C₁₈-alkylamidoethyltrimethylammonium ether sulfates, C₈- to C₁₈-alkylmethyl-amine oxides and C₈- to C₁₈-alkylaminoethyldimethylamine oxides.

In various embodiments the composition according to the invention contains from 0.01 to 5, especially preferably from 0.05 to 1, percent by weight of at least one preservative. Suitable preservatives are the substances listed in International Cosmetic Ingredient Dictionary and Handbook, 9^{th} Edition, with the function “Preservatives”, such as phenoxyethanol, benzyl-p-hydroxybenzoic acid, butyl-p-hydroxybenzoic acid, ethyl-p-hydroxybenzoic acid, isobutyl-p-hydroxy-benzoic acid, isopropyl-p-hydroxybenzoic acid, methyl-p-hydroxybenzoic acid, propyl-p-hydroxybenzoic acid, iodopropinylbutylcarbamate, methyldibromoglutaronitrile, and DMDM hydantoin.

In a preferred embodiment of the invention the composition according to the invention also includes at least one pigment. This pigment can be a colored pigment, which imparts a color effect to the product mass or the hair or it can be a luster effect pigment, which imparts a luster effect to the hair or the product mass. The color and luster effects on the hair are preferably temporary, i.e. they are maintained until the next hair washing and can be removed by washing the hair again with conventional shampoos. The pigments are present in the product mass in undissolved form. They can be contained in an amount of 0.01 to 25 percent by weight, especially preferably from 5 to 15 percent by weight. The preferred particle size amounts to from 1 to 200 μm, especially from 3 to 150 μm, especially preferably from 10 to 100 μm. The pigments are practically insoluble coloring agents and can be inorganic or organic. Also inorganic-organic mixed pigments may be used. Inorganic pigments are preferable. The advantage of the inorganic pigments is their outstanding light-resistance, weather-resistance and temperature-resistance. The inorganic pigments can be of natural origin, for example chalk, ocher, umber, green earth, burnt siena or graphite. The pigments can be white pigments, such as titanium dioxide or zinc oxide; black pigments, such as iron oxide black; fancy or multi-colored pigments, such as ultramarine or iron oxide red; lustrous pigments, metal effect pigments, pearlescent pigments as well as fluorescent or phosphorescent pigments. Preferably at least one pigment is a colored, non-white pigment. Metal oxides, metal hydroxides and metal oxide hydrates, mixed phase pigments, sulfur-containing silicates, metal sulfides, complex metallo-cyanides, metal sulfates, metal chromates and metal molybdates and metals themselves (bronze pigments) are suitable. In particular, titanium dioxide (C.I. 77891), black iron oxide (C.I. 77499), yellow iron oxide (C.I. 77492), red and brown iron oxide (C.I. 77491), manganese violet (C.I. 77742), ultramarine (sodium aluminum sulfosilicate, C.I. 77007, Pigment Blue 29), chromium oxide hydrate (C.I. 77289), Iron Blue (Ferric ferrocyanide, C.I. 77510) and carmine (cochineal), are all suitable pigments.

Pigments based on mica and/or isinglass, which are coated with a metal oxide or metal oxychloride, such as titanium dioxide or bismuth oxychloride and if necessary other color-imparting materials, such as iron oxides, iron blue, ultramarine, carmine, etc, whose colors can be modified by changing the thickness of the coating, are especially preferred. Pigments of this sort are marketed, for example, under the trademark, Rona®, Colorona®, Dichrona® and Timiron® by the firm, Merck, Germany.

Organic pigments are, for example, the natural pigments, Sepia, gamboge, charcoal, Kasseler brown, indigo, chlorophyl and other plant pigments. Synthetic organic pigments include, for example, azo pigments, anthraquinoid pigments, indigoid pigments, dioxazine, quinacridone, phthalocyanine isoindolinone pigments, perylene pigments, perinone pigments, metal complex pigments, alkali blue pigments and diketopyrrolopyrrole pigments.

In one embodiment the composition according to the invention contains from 0.01 to 10, especially preferably from 0.05 to 5 percent by weight, of at least one particulate material. Suitable materials are, e.g., those material, which are solid at room temperature (25° C.) and are present in the form of particles. Some suitable materials are silica, silicates, aluminates, clay, mica, salt, especially inorganic metal salts, metal oxides, e.g. titanium dioxide, minerals and polymer particles.

The particles are present in the composition in undissolved form, preferably in stable dispersed form, and can be deposited in solid form in the hair after application to the hair and evaporation of solvent. A stable dispersion can be achieved, when the composition is provided with a flow point, which is sufficiently large, in order to prevent the precipitation of the solid particles. A sufficient flow point can be established by using a suitable gel former in a sufficient quantity.

The preferred particulate materials are silica (silica gel, silicon dioxide) and metal salts, especially inorganic metal salts, but silica is especially preferred. Metal salts are, e.g., alkali or alkaline earth halides, such as sodium chloride or potassium chloride, alkali or alkaline earth sulfates, such as sodium sulfate or magnesium sulfate.

Water or a water/alcohol mixture is used as solvent in the gel according to the invention. The alcohol content can be up to 50 percent by weight. The alcohols can especially be lower alcohols with 1 to 4 carbon atoms, such as ethanol and isopropanol, which are suitable for cosmetic purposes. The composition according to the invention can have a pH in a range from 2.0 to 9.5. A pH range between 4 and 8 is particularly preferred. Organic solvents or mixtures of solvents with a boiling point under 400° C. can be contained in the composition according to the invention in an amount of from 0.1 to 15 percent by weight, preferably from 1 to 10 percent by weight, as additional co-solvents. Polyols, such as glycerol, ethylene glycol and propylene glycol, are especially suitable.

The composition according to the invention can be used in different application forms, e.g. as a gel for dispensing from a tube, as a liquid gel, as a sprayable gel with a suitable spraying apparatus br as a foam gel with a suitable apparatus for foaming.

The viscosity preferably amounts to at least 250 mPa s, especially from 500 to 50,000 mPa s, especially preferably from 1,000 to 15,000 mPa s or from 5,000 to 15,000 mPa s, as measured with a Haake Viscotester 550, measurement body SV (DIN 53019) at 25° C. and a shear rate of 12.9 s⁻¹.

A preferred embodiment of the hairstyling gel according to the invention contains
(A) from 0.5 to 10 percent by weight of at least one gel former, selected from the group consisting of acrylate/C10-30 alkylacrylate crosspolymers, carbomers, xanthan gum and acrylate/ceteth-20 itaconate copolymer;
(B) from 3 to 15 percent by weight of at least one UV filter, which is an organic compound with at least one benzimidazole group and at least one sulfonic acid group and its salts; and
(C) from 0.1 to 15 percent by weight of at least one hair-fixing polymer, which is at least one anionic and/or nonionic polymer.

The following examples should clearly illustrate the subject matter of the invention. The stated polymer content relates, when not otherwise indicated, to the solids content.

### EXAMPLES

The following products were employed in the examples:
HELIOPAN® Hydro: 2-phenyl-1H-benzimidazol-5-sulfonic acid,
HELIOPAN® AP: 2,2′-(1,4-phenylen)-bis-(1H-benzimidazol-4,6-disulfonic acid),
ULTREZ® 21: Acrylate/C10-30 Alkylacrylate crosspolymer,
AQUAFLEX® SF 40: vinyl pyrrolidone/vinyl caprolactam/dimethyl-aminopropylacrylamide copolymer,
LUVISKOL® VA 64: VP/VA copolymer,
   STRUCTURE® 3001: Acrylates/Ceteth-20 Itaconate Cop[olymer,

ACULYN® 48: PEG-150/Stearyl alcohol/SMDI copolymer, 19% in water.

EXAMPLE 1
Hairstyling Gel -- Normal Hold

| Ingredients | % by weight |
|---|---|
| ULTREZ ® 21 | 2.0 |
| Polyvinyl pyrrolidone K 90 | 1.8 |
| HELIOPAN ® Hydro | 2.0 |
| HELIOPAN ® AP | 2.0 |
| Aminomethylpropanol 95% | 3.0 |
| PEG-40 Hydrogenated Castor Oil | 0.2 |
| Perfume | 0.2 |
| Water | To 100 |

EXAMPLE 2
Hairstyling Gel -- Strong Hold

| Ingredients | % by weight |
|---|---|
| Carbomer (CARBOPOL ®) | 2.0 |
| Vinyl acetate/crotonate copolymer | 2.5 |
| (LUVISET ® CA 66) | |
| HELIOPAN ® Hydro | 8.0 |
| Aminomethylpropanol 95% | 4.0 |
| PEG-40 Hydrogenated Castor Oil | 0.2 |
| Panthenol | 0.1 |
| Perfume | 0.2 |
| Water | To 100 |

EXAMPLE 3
Hairstyling Gel -- Strong Hold

| Ingredients | % by weight |
|---|---|
| Xanthen gum (AMAZE ® XT) | 1.5 |
| AQUAFLEX ® SF 40 | 2.7 |
| HELIOPAN ® Hydro | 6.0 |
| HELIOPAN ® AP | 6.0 |
| Aminomethylpropanol 95% | 3.6 |
| PEG-25 PABA | 0.5 |
| PEG-40 Hydrogenated Castor Oil | 0.2 |
| Panthenol | 0.1 |
| Perfume | 0.2 |
| Water | To 100 |

EXAMPLE 4
Hairstyling Gel -- Normal Hold

| Ingredients | % by weight |
|---|---|
| ULTREZ ® 21 | 2.0 |
| Glycerol | 5.2 |
| Polyethylene glycol | 4.0 |
| HELIOPAN ® AP | 6.0 |
| Aminomethylpropanol 95% | 3.6 |
| Vinyl pyrrolidone/vinyl acetate | 3.0 |
| copolymer | |
| Water | To 100 |

EXAMPLE 5
Hairstyling Gel -- Spray

| Ingredients | % by weight |
|---|---|
| Xanthen gum (AMAZE ® XT) | 1.0 |
| Vinyl pyrrolidone/vinyl acetate | 1.5 |
| copolymer | |
| HELIOPAN ® Hydro | 2.0 |
| HELIOPAN ® AP | 2.0 |
| Aminomethylpropanol 95% | 1.2 |
| Perfume | 0.15 |
| Ethanol | 10.0 |
| Water | To 100 |

EXAMPLE 6
Hairstyling Gel -- Spray gel

| Ingredients | % by weight |
|---|---|
| Ammonium acryloyoldimethyltaurate/ | 1.0 |
| Vinyl pyrrolidone copolymer (ARISTOFLEX ® AVC) | |
| Vinyl pyrrolidone/vinyl acetate | 3.0 |
| Ethanol | 18.0 |
| HELIOPAN ® Hydro | 8.0 |
| Aminomethylpropanol 95% | 2.7 |
| PEG-40 | 0.2 |
| Perfume | 0.2 |
| Water | To 70 |

EXAMPLE 7
Hairstyling Gel -- Spray gel

| Ingredients | % by weight |
|---|---|
| STRUCTURE ® 3001 | 3.5 |
| Vinyl pyrrolidone/vinyl acetate | 3.0 |
| Ethanol | 8.0 |
| HELIOPAN ® Hydro | 2.0 |
| HELIOPAN ® AP | 2.0 |
| Aminomethylpropanol 95% | 2.8 |
| PEG-40 | 0.2 |
| ACULYN ® 48 | 0.5 |
| Perfume | 0.2 |
| Water | To 70 |

EXAMPLE 8
Hairstyling Gel -- Liquid

| Ingredients | % by weight |
|---|---|
| Carbomer (CARBOPOL ®) | 1.0 |
| Vinyl acetateicrotonate copolymer | 1.5 |
| (LUVISET ® CA 66) | |
| Propylene glycol | 3.8 |
| Hydroxypropyl guar | 0.3 |
| HELIOPAN ® Hydro | 6.0 |
| HELIOPAN ® AP | 6.0 |
| Aminomethylpropanol 95% | 3.8 |
| PEG-40 Hydrogenated Castor Oil | 0.18 |
| PPG-1-PEG-9 Lauryl Glycol Ether | 0.18 |
| Perfume | 0.15 |
| Ethanol | 16.5 |
| Water | To 100 |

EXAMPLE 9
Hairstyling Gel -- Liquid

| Ingredients | % by weight |
|---|---|
| Carbomer (CARBOPOL ®) | 1.5 |
| PEG-12 Dimethicone | 1.5 |
| (Dow Corning Surfactant 193) | |
| STRUCTURE ® 3001 | 2.0 |
| HELIOPAN ® AP | 6.0 |
| Aminomethylpropanol 95% | 3.7 |
| PPG-1-PEG-9 lauryl glycol ether | 0.2 |
| Perfume | 0.15 |
| Hydroxyethyl cellulose | 0.4 |
| Ethanol | 16.5 |
| Water | To 100 |

EXAMPLE 10
Hairstyling Gel

| Ingredients | % by weight |
|---|---|
| Carbomer (CARBOPOL ®) | 2.0 |
| LUVISET ® CA 66 | 2.5 |
| HELIOPAN ® Hydro | 1.0 |
| Aminomethylpropanol 95% | 2.0 |
| PEG-40 Hydrogenated Castor Oil | 0.2 |
| Panthenol | 0.1 |
| Perfume | 0.2 |
| Water | To 100 |

The disclosure in German Patent Application 102004034265.2 of Jul. 15, 2004 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in hairstyling gels with light protective action for skin and hair, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A hairstyling gel containing a combination of
(A) at least one gel former;
(B) more than two percent by weight of at least one UV filter and
(C) at least one hair-fixing polymer.

2. The gel as defined in claim 1, having a sun protective factor of at least six.

3. The gel as defined in claim 1, containing from 0.5 to 20 percent by weight of said at least one gel former, from 0.1 to 15 percent by weight of said at least one hair-fixing polymer and/or from 3 to 15 percent by weight of said at least one UV filter.

4. The gel as defined in claim 1, having a viscosity of at least 250 mPa s at 25° C. and a shear rate of 12.9 s⁻¹.

5. The gel as defined in claim 1, wherein said at least one gel former is
at least one copolymer of at least one first monomer and at least one second monomer, wherein said at least one second monomer is selected from the group consisting of esters of acrylic acid and ethoxylated fatty alcohols and said at least one first monomer is selected from the group consisting of acrylic acid and methacrylic acid;
at least one cross-linked polyacrylic acid;
at least one cross-linked copolymer of at least one first monomer compound and at least one second monomer compound, wherein said at least one first monomer compound is acrylic acid and/or methacrylic acid and said at least one second monomer compound is selected from the group consisting of esters of acrylic acid with C₁₀- to C₃₀-alcohols;
at least one copolymer of at least one first monomer ingredient and at least one second monomer ingredient, wherein said at least one first monomer ingredient is acrylic acid and/or methacrylic acid and wherein said at least one second monomer ingredient is selected from the group consisting of esters of itaconic acid and ethoxylated fatty alcohols;
at least one copolymer of at least one first monomeric compound, at least one second monomeric compound and a C₁- to C₄-aminoalkyl acrylate, wherein said at least one first monomeric compound is acrylic acid and/or methacrylic acid, said at least one second monomeric compound is selected from the group consisting of itaconic acid and ethoxylated C₁₀- to C₃₀-alcohols;
at least one copolymer of at least two of acrylic acid, methacrylic acid, acrylic acid esters and methacrylic acid esters;
at least one copolymer of vinyl pyrrolidone and ammonium acryloyldimethyltaurate;
at least one copolymer of ammonium acryloyldimethyltaurate and at least one member selected from the group consisting of methacrylic acid and ethoxylated fatty alcohols;
at least one glyceryl polyacrylate;
at least one glyceryl polymethacrylate;
at least one copolymer of styrene and at least one C₂-, C₃- or C₄-alkylene;
at least one polyurethane;
at least one copolymer of polyethylene oxide, fatty alcohols and saturated methylenediphenyl diisocyanate;
at least one hydroxypropyl starch phosphates;
at least one polyacrylamide;
at least one copolymer of maleic acid anhydride and methyl vinyl ether cross-linked with decadiene; and
at least one natural thickener ingredient selected from the group consisting of carob bean seed meal; guar gum; xanthen, dehydroxyxanthen; carrageenan, karaya gum; hydrolyzed corn starch; hydroxyethyl cellulose, hydroxypropyl cellulose and hydroxypropyl guar.

6. The gel as defined in claim 1, wherein the at least one UV filter contains at least one benzimidazole or benzoxazole group and at least one sulfonic acid group.

7. The gel as defined in claim 1, wherein the at least one UV filter comprises a combination of at least one UV-A filter and at least one UV-B filter.

8. The gel as defined in claim 1, wherein said at least one UV filter is selected from the group consisting of phenylbenzimidazole sulfonic acid, salts of phenylbenzimidazole sulfonic acid, phenyldibenzimidazole tetrasulfonic acid and salts of phenyldibenzimidazole tetrasulfonic acid.

9. The gel as defined in claim 1, wherein said at least one hair-fixing polymer is at least one anionic polymer, at least one cationic polymer, at least one zwitterionic and/or amphoteric polymer and/or at least one nonionic polymer;
wherein said at least one anionic polymer is selected from the group consisting of terpolymers of acrylic acid, ethyl acrylate and N-t-butyl-acrylamide; cross-linked vinyl acetate/crotonic acid copolymers; uncrosslinked vinyl acetate/crotonic acid copolymers; terpolymers from tert.-butylacrylate, ethyl acrylate and methacrylic acid; sodium polystyrene sulfonate; copolymers of vinyl acetate, crotonic acid and vinyl propionate; copolymers of vinyl acetate, crotonic acid and vinyl neodecanoate; aminomethyl propanol acrylate copolymers; copolymers of vinyl pyrrolidone and at least one other monomer, which is at least one of acrylic acid, methacrylic acid, acrylic acid esters and methacrylic acid esters; copolymers of methyl vinyl ether and maleic acid monoalkyl esters; aminomethylpropanol salts of copolymers of allyl methacrylate and at least one further monomer, which is at least one of acrylic acid, methacrylic acid, acrylic acid esters and methacrylic acid esters; cross-linked copolymers of ethyl acrylate and methacrylic acid; copolymers of vinyl acetate, mono-n-butylmaleate and isobornyl acrylate; copolymers of two or more monomers, which are at least two of acrylic acid, methacrylic acid, acrylic acid esters and methacrylic acid esters; copolymers of octyl acrylamide and at least one monomer, which is at least one of acrylic acid, methacrylic acid, acrylic acid esters and methacrylic acid esters; and polyesters of diglycol, cyclohexanedimethanol, isophthalic acid and sulfoisophthalic acid;
wherein said at least one cationic polymer is selected from the group consisting of cationic cellulose derivatives comprising hydroxyethyl cellulose and diallyldimethyl ammonium chloride; cationic cellulose derivates comprising hydroxyethyl cellulose and with trimethyl ammonium substituted epoxide; poly(dimethyldiallylammonium chloride); copolymers of acrylamide and poly(dimethyldiallylammonium chloride); quaternary ammonium polymers formed by reaction of diethyl sulfate and a copolymer of vinyl pyrrolidone and dimethylaminoethyl methacrylate; quaternary ammonium polymers of methyl-vinylimidazolium chloride and vinyl pyrrolidone; polyquaternium-35; polymers from trimethylammonium-ethyl-methacrylate chloride; polyquaternium-57; substituted dimethylpolysiloxanes terminated with quarternary ammonium groups; copolymers of vinyl pyrrolidone, dimethylaminopropylmethacrylamide and methacryloylaminopropyllauryldimethylammonium chloride; chitosan; chitosan salts; hydroxyalkylchitosans; salts of the hydroxyalkylchitosans; alkyl-hydroxyalkylchitosans; salts of the alkyl-hydroxyalkylchitosans; N-hydroxyalkyl-chitosanalkyl ethers; N-hydroxyalkyl-chitosanbenzyl ethers; copolymers of vinyl caprolactam, vinyl pyrrolidone and dimethylaminoethylmethacrylate; copolymers of vinyl pyrrolidone and dimethylaminoethylmethacrylate; copolymers of vinyl pyrrolidone, vinyl caprolactam and dimethylaminopropylacrylamide; polyesters built up from hydroxyl acids substituted with at least one quaternary ammonium group;
wherein said at least one zwitterionic and/or amphoteric polymer is selected from the group consisting of copolymers of octylacrylamide, acrylic acid, butylaminoethylmethacrylate, methylmethacrylate and hydroxypropyl-methacrylate; copolymers of lauryl acrylate, stearyl acrylate, ethylaminoxide methacrylate and at least one of acrylic acid, methacrylic acid, acrylic acid esters and methacrylic acid esters; copolymers of methacryloylethyl betaine and at least one of acrylic acid, methacrylic acid, acrylic acid esters and methacrylic acid esters; copolymers of acrylic acid, methacrylate and methacrylamido-propyltrimethyl ammonium chloride; polymers made from quaternary crotonic betaines and polymers made from quaternary crotonic betaine esters; and
wherein said at least one nonionic polymer is selected from the group consisting of polyvinyl pyrrolidone; polyvinyl caprolactam; vinyl pyrrolidone/vinyl acetate copolymers; polyvinyl alcohols; isobutylene/ethylmaleimid/hydroxy-ethylmaleimide copolymers; copolymers of vinyl pyrrolidone, vinyl acetate and vinyl propionate and terpolymers of vinyl pyrrolidone, methacrylamide and vinyl imidazole.

10. The gel as defined in claim 1, which is optically clear.

11. The gel as defined in claim 1, in an optically clear emulsified form and containing at least one perfume oil.

12. The gel as defined in claim 1, in the form of a liquid gel or in the form of a sprayable gel, said sprayable gel being sprayable by means of a mechanical device.

13. The gel as defined in claim 1, containing
(A) from 0.5 to 10 percent by weight of said at least one gel former, said at least one gel former being selected from the group consisting of acrylate/C10-30 alklyacrylate crosspolymer, carbomers, xanthen gum and acrylates/ceteth-20 itaconate copolymer;
(B) from 3 to 15 percent by weight of said at least one UV filter, wherein said at least one UV filter comprises at least one organic compound with least one functional group and said at least one functional group is selected from the group consisting of benzimidazole groups, benzimidazole salt groups, a sulfonic acid group and sulfonic acid salt groups; and
(C) from 0.1 to 15 percent by weight of said at least one hair-fixing polymer, wherein said at least one hair-fixing polymer is at least one anionic and/or nonionic polymer.
